(19) United States
(12) Patent Application Publication
(10) Pub. No.: US 2024/0369749 A1
(43) Pub. Date: Nov. 7, 2024

(54) OPTICAL FILTER AND STERILIZATION DEVICE

(71) Applicant: NIPPON ELECTRIC GLASS CO., LTD., Otsu-shi, Shiga (JP)

(72) Inventors: Tomoyoshi NAKAMURO, Otsu-shi (JP); Tsutomu IMAMURA, Otsu-shi (JP)

(21) Appl. No.: 18/687,408

(22) PCT Filed: Sep. 2, 2022
(86) PCT No.: PCT/JP2022/033091

§ 371 Date: Feb. 28, 2024

(30) Foreign Application Priority Data
Sep. 14, 2021 (JP) 2021-149557

(51) Int. Cl. G02B 5/28; A61L 2/10; A61L 2/26

(52) U.S. Cl. CPC G02B 5/28; A61L 2/10; A61L 2/26; A61L 2202/11

(57) ABSTRACT

Provided is an optical filter in which a dielectric multi-layer film is less likely to separate from a transparent substrate. An optical filter 1 includes: a transparent substrate 2 made of glass; and a dielectric multi-layer film 3 provided on the transparent substrate 2 and containing hafnium oxide, wherein a surface of the transparent substrate 2 on the dielectric multi-layer film 3 side has an arithmetic mean height Sa of 0.22 nm or less.

### TECHNICAL FIELD

The present invention relates to optical filters and sterilization devices using the optical filters.

### BACKGROUND ART

Optical filters capable of selectively transmitting light in a specific wavelength range are widely used in various applications. As an optical filter as just described, there is known a bandpass filter in which a dielectric film is used.

Meanwhile, there has been recently proposed a sterilization device that allows ultraviolet radiation to act on DNA in cells of organisms to be sterilized, such as bacteria, without harming human cells, to selectively inactivate the organisms to be sterilized. In the sterilization device, an optical filter (interference filter) is used which includes a dielectric multi-layer film where hafnium oxide layers and silicon oxide layers are alternately layered one on top of another in order to transmit, among light emitted from a light source, light in a wavelength range of 190 nm to 230 nm and block light of wavelengths other than the above wavelength range.

For example, Patent Literature 1 below discloses a microbial inactivation treatment device that subjecting microorganisms to be treated to inactivation treatment by irradiating them through an optical filter with light emitted from a light source. Patent Literature 1 describes that when the light emitted from the light source enters the optical filter at an angle of incidence of 0°, the optical filter transmits at least part of ultraviolet radiation with wavelengths of not less than 190 nm and not more than 230 nm and at least part of ultraviolet radiation with wavelengths of more than 230 nm and not more than 237 nm and blocks transmission of ultraviolet radiation out of a wavelength range of not less than 190 nm and not more than 237 nm.

### CITATION LIST

### Patent Literature

### [PTL 1]

### JP-A-2019-115525

### SUMMARY OF INVENTION

### Technical Problem

Conventionally, an optical filter has been used in which a dielectric multi-layer film with hafnium oxide layers and silicon oxide layers alternately layered one on top of another is provided on a transparent substrate.

In the case of use of a dielectric multi-layer film containing hafnium oxide, an optical filter may be produced by forming a dielectric multi-layer film on a transparent substrate and then subjecting the obtained laminate product to heat treatment in order to crystallize hafnium oxide.

However, the conventional optical filter may cause separation at the interface between the transparent substrate and the dielectric multi-layer film after the heat treatment.

An object of the present invention is to provide an optical filter in which a dielectric multi-layer film is less likely to separate from a transparent substrate. Furthermore, the present invention also has an object of providing a sterilization device using the above-described optical filter.

### Solution to Problem

The inventors found that the above problem can be solved by 1) controlling the arithmetic mean height Sa of a surface of a transparent substrate on a dielectric multi-layer film side, 2) controlling the arithmetic mean height Sa of a surface of the dielectric multi-layer film on the side opposite to the transparent substrate side or 3) purposefully placing a layer between the transparent substrate and the dielectric multi-layer film, and completed the invention.

An optical filter according to the present invention includes: a transparent substrate made of glass; and a dielectric multi-layer film provided on the transparent substrate and containing hafnium oxide, wherein a surface of the transparent substrate on the dielectric multi-layer film side has an arithmetic mean height Sa of 0.22 nm or less.

An optical filter according to the present invention includes: a transparent substrate made of glass; and a dielectric multi-layer film provided on the transparent substrate and containing hafnium oxide, wherein a surface of the dielectric multi-layer film on a side opposite to the transparent substrate side has an arithmetic mean height Sa of 1.00 nm or less.

An optical filter according to the present invention includes: a transparent substrate made of glass; a dielectric multi-layer film containing hafnium oxide; and an adhesion layer provided between the transparent substrate and the dielectric multi-layer film.

In the optical filter according to the present invention, the adhesion layer is preferably a layer containing silicon oxide.

A sterilization device according to the present invention is a sterilization device capable of subjecting microorganisms to be treated to inactivation treatment, the sterilization device including: a light source a wavelength of light emitted from which is in a wavelength range of 190 nm to 230 nm; and the above-described optical filter.

The sterilization device according to the present invention preferably includes a retainer that retains the optical filter in a curved form.

### Advantageous Effects of Invention

The present invention enables provision of: an optical filter in which a dielectric multi-layer film is less likely to separate from a transparent substrate; and a sterilization device using the optical filter.

### BRIEF DESCRIPTION OF DRAWINGS

FIG. 1 is a schematic cross-sectional view showing an optical filter according to a first embodiment of the present invention.

FIG. 2 is a schematic cross-sectional view showing an optical filter according to a second embodiment of the present invention.

FIG. 3 is a schematic view showing a sterilization device according to a first embodiment of the present invention.

FIG. 4 is a schematic view showing a sterilization device according to a second embodiment of the present invention.

### DESCRIPTION OF EMBODIMENTS

Hereinafter, a description will be given of preferred embodiments. However, the following embodiments are merely illustrative and the present invention is not limited to the following embodiments. Throughout the drawings, members having substantially the same functions may be referred to by the same reference characters.

### Optical Filter

### First Embodiment

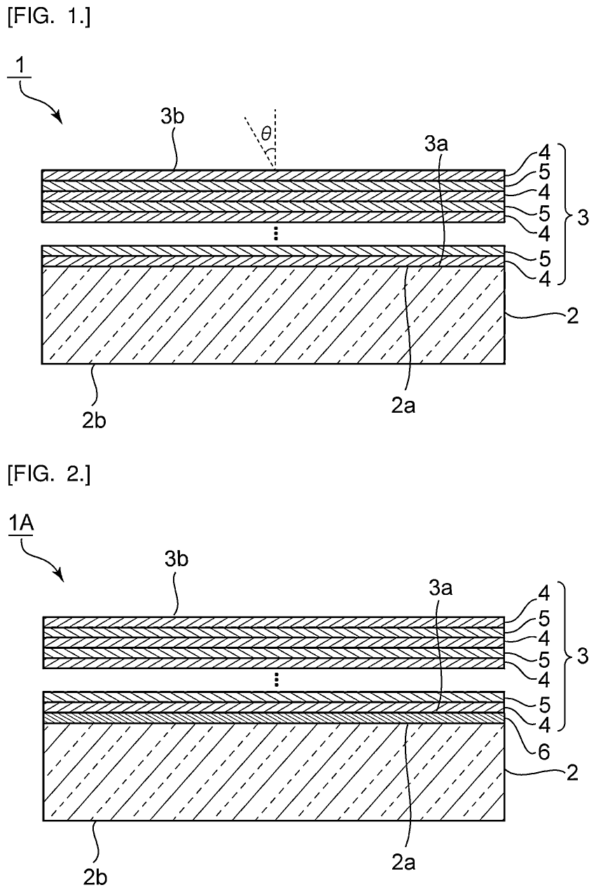

FIG. 1 is a schematic cross-sectional view showing an optical filter according to a first embodiment of the present invention.

The optical filter 1 shown in FIG. 1 includes a transparent substrate 2 and a dielectric multi-layer film 3. In the optical filter 1, the dielectric multi-layer film 3 is provided on the transparent substrate 2.

The transparent substrate 2 has a first principal surface 2a and a second principal surface 2b. The first principal surface 2a and the second principal surface 2b are surfaces of the transparent substrate 2 opposed to each other. The dielectric multi-layer film 3 has a first principal surface 3a and a second principal surface 3b. The first principal surface 3a and the second principal surface 3b are surfaces of the dielectric multi-layer film 3 opposed to each other.

The first principal surface 2a of the transparent substrate 2 is a surface thereof on the dielectric multi-layer film 3 side. The second principal surface 2a of the transparent substrate 2 is one of both the principal surfaces of the optical filter 1. The first principal surface 3a of the dielectric multi-layer film 3 is a surface thereof on the transparent substrate 2 side. The second principal surface 3b of the dielectric multi-layer film 3 is the other principal surface of the optical filter 1 (a principal surface thereof on the side opposite to the transparent substrate 2 side).

The dielectric multi-layer film 3 is provided immediately on the first principal surface 2a of the transparent substrate 2. The transparent substrate 2 is provided immediately on the first principal surface 3a of the dielectric multi-layer film 3. The first principal surface 2a of the transparent substrate 2 and the first principal surface 3a of the dielectric multi-layer film 3 are in contact.

The transparent substrate 2 has a rectangular plate-like shape. However, the shape of the transparent substrate 2 is not particularly limited. The transparent substrate 2 may have any other shape, such as, for example, a disc-like shape.

The transparent substrate 2 is made of glass. The transparent substrate 2 is a transparent glass substrate. The transparent substrate 2 preferably has an average light transmittance of 80% or more in a ultraviolet wavelength range of 220 nm to 225 nm.

Heretofore known glasses used in optical filters can be used as the glass constituting the transparent substrate 2. Example of the glass include quartz glass and borosilicate glass. The quartz glass may be synthesized quartz glass or fused quartz glass. The borosilicate glass preferably contains, in terms of % by mass as its glass composition, 55% to 75% Si02, 1.0% to 10% Al₂O₃, 10% to 30% B₂O₃, 0% to 5% Cao, 0% to 5% BaO, and 1.0% to 15% Li₂O+Na₂O+K₂O, and more preferably further contains 0% to 0.001% TiO₂, 0% to 0.001% Fe₂O₃, and 0.5% to 2.0% F.

The thickness of the transparent substrate 2 is not particularly limited and can be appropriately selected according to a desired light transmittance, the form of the optical filter 1 in use, such as whether the optical filter 1 is used in a curved form or in a non-curved form, and so on. The thickness of the transparent substrate 2 may be, for example, about 2 μm to about 30 mm. In the case of use of the optical filter 1 in a curved form, the thickness of the transparent substrate 2 is preferably not less than 2 μm, preferably not more than 0.2 mm, and more preferably not more than 0.1 mm. In the case of use of the optical filter 1 in a non-curved form (in the case of use thereof in a flat form), the thickness of the transparent substrate 2 is preferably not less than 0.1 mm and preferably not more than 30 mm.

The dielectric multi-layer film 3 is a multi-layer film including high-refractive index films 4 and low-refractive index films 5. The high-refractive index films 4 have a higher refractive index than the low-refractive index films 5, whereas the low-refractive index films 5 have a lower refractive index than the high-refractive index films 4. In the dielectric multi-layer film 3, the high-refractive index films 4 and the low-refractive index films 5 are alternately layered one on top of another in a direction of the thickness of the dielectric multi-layer film 3.

In this embodiment, the dielectric multi-layer film 3 is made up by alternately layering the high-refractive index films 4 and the low-refractive index films 5 in this order on the first principal surface 2a of the transparent substrate 2. In this embodiment, a high-refractive index film 4 is disposed on the first principal surface 2a of the transparent substrate 2. In this embodiment, the first principal surface 3a of the dielectric multi-layer film 3 is a surface of the high-refractive index film 4.

In this embodiment, the second principal surface 3b of the dielectric multi-layer film 3 is a surface of a high-refractive index film 4. However, the second principal surface 3b of the dielectric multi-layer film 3 may be a surface of a low-refractive index film. In other words, the outermost layer of the dielectric multi-layer film may be a high-refractive index film or a low-refractive index film.

The high-refractive index films 4 are made of hafnium oxide and are films consisting primarily of hafnium oxide. The high-refractive index films 4 are hafnium oxide layers. Since the high-refractive index films 4 contain hafnium oxide, the dielectric multi-layer film 3 contains hafnium oxide.

As used herein, the above term “film consisting primarily of” and the term “layer consisting primarily of” to be described hereinafter mean that the film or the layer contains the relevant component in an amount of 50% by mass or more. In the “film consisting primarily of” and the “layer consisting primarily of”, the film or the layer preferably contains the relevant component in an amount of 80% by mass or more and more preferably contains it in an amount of 90% by mass or more. Needless to say, the film or the layer may contain the relevant component in an amount of 100% by mass.

In this embodiment, the low-refractive index films 5 are made of silicon oxide and are films consisting primarily of silicon oxide. The low-refractive index films 5 are silicon oxide layers. However, the low-refractive index films 5 may be films consisting primarily of aluminum oxide, zirconium oxide, tin oxide, magnesium fluoride or silicon nitride. These materials for the low-refractive index films 5 may be used singly or in a combination of a plurality of them.

The arithmetic mean height Sa of the surface of the transparent substrate 2 on the dielectric multi-layer film 3 side (the first principal surface 2a of the transparent substrate 2) is preferably not more than 0.22 nm, more preferably not more than 0.20 nm, and still more preferably not more than 0.15 nm. When the above arithmetic mean height Sa is not more than the above upper limit, the adhesion force between the transparent substrate 2 and the dielectric multi-layer film 3 can be increased and, therefore, the separation of the dielectric multi-layer film 3 from the transparent substrate 2 can be effectively prevented even when they are subjected to heat treatment or the optical filter 1 is used in a curved form. The arithmetic mean height Sa of the surface of the transparent substrate 2 on the dielectric multi-layer film 3 side (the first principal surface 2a of the transparent substrate 2) may be not less than 0.05 nm or not less than 0.10 nm. As thus far described, the arithmetic mean height Sa of the first principal surface 2a of the transparent substrate 2, which is the surface of the transparent substrate 2 on the dielectric multi-layer film 3, is preferably not less than 0.05 nm, more preferably not less than 0.10 nm, not more than 0.22 nm, preferably not more than 0.20 nm, and more preferably not more than 0.15 nm.

The arithmetic mean height Sa of the surface of the dielectric multi-layer film 3 on the side opposite to the transparent substrate 2 side (the second principal surface 3b of the dielectric multi-layer film 3) is preferably not more than 1.00 nm, more preferably not more than 0.70 nm, and still more preferably not more than 0.50 nm. When the above arithmetic mean height Sa is not more than the above upper limit, the adhesion force between the transparent substrate 2 and the dielectric multi-layer film 3 can be increased and, therefore, the separation of the dielectric multi-layer film 3 from the transparent substrate 2 can be effectively prevented even when they are subjected to heat treatment or the optical filter 1 is used in a curved form. The arithmetic mean height Sa of the surface of the dielectric multi-layer film 3 on the side opposite to the transparent substrate 2 side (the second principal surface 3b of the dielectric multi-layer film 3) may be not less than 0.05 nm or not less than 0.10 nm. As thus far described, the arithmetic mean height Sa of the second principal surface 3b of the dielectric multi-layer film 3, which is the surface of the dielectric multi-layer film 3 on the side opposite to the transparent substrate 2 side, is preferably not less than 0.05 nm, more preferably not less than 0.10 nm, preferably not more than 1.00 nm, more preferably not more than 0.70 nm, and still more preferably not more than 0.50 nm.

In the present invention, it is preferred that the arithmetic mean height Sa of the surface of the transparent substrate 2 on the dielectric multi-layer film 3 side is not more than the above upper limit and the arithmetic mean height Sa of the surface of the dielectric multi-layer film 3 on the side opposite to the transparent substrate 2 side is not more than the above upper limit. In this case, the effects of the present invention can be more effectively exerted.

The root-mean-square height Sq of the surface of the transparent substrate 2 on the dielectric multi-layer film 3 side (the first principal surface 2a of the transparent substrate 2) is preferably not more than 0.25 nm, more preferably not more than 0.23 nm, and still more preferably not more than 0.20 nm. When the above root-mean-square height Sq is not more than the above upper limit, the adhesion force between the transparent substrate 2 and the dielectric multi-layer film 3 can be increased and, therefore, the separation of the dielectric multi-layer film 3 from the transparent substrate 2 can be effectively prevented even when they are subjected to heat treatment or the optical filter 1 is used in a curved form. The root-mean-square height Sq of the surface of the transparent substrate 2 on the dielectric multi-layer film 3 side (the first principal surface 2a of the transparent substrate 2) may be not less than 0.05 nm or not less than 0.10 nm. As thus far described, the root-mean-square height Sq of the first principal surface 2a of the transparent substrate 2, which is the surface of the transparent substrate 2 on the dielectric multi-layer film 3 side, is preferably not less than 0.05 nm, more preferably not less than 0.10 nm, preferably not more than 0.25 nm, more preferably not more than 0.23 nm, and still more preferably not more than 0.20 nm.

Each of the above-described arithmetic mean heights Sa and the above-described root-mean-square height Sq can be measured with a white-light interferometer (for example, “NewView 7300” manufactured by Zygo Corporation) in conformity with ISO 25178.

Furthermore, each of the above-described arithmetic mean heights Sa and the above-described root-mean-square height Sq can be reduced, for example, by appropriately adjusting the conditions of polishing of the surface of the transparent substrate or removing foreign matters (such as glass powder) attached in minute amounts to the surface of the transparent substrate.

The optical filter 1 is a bandpass filter designed to selectively transmit light in a specific wavelength range by optical interference.

In the optical filter 1, the minimum value of the spectral transmittances at wavelengths of 220 nm to 225 nm at an angle of incidence of 0° is preferably not less than 50%, more preferably not less than 60%, and still more preferably not less than 70%. When the minimum value of the above spectral transmittances is not less than the above lower limit, the optical filter 1 when used for a sterilization device can efficiently transmit ultraviolet radiation useful for sterilization treatment.

In the optical filter 1, the minimum value of the spectral transmittances at wavelengths of 220 nm to 225 nm at an angle of incidence of 30° is preferably not less than 25%, more preferably not less than 35%, and still more preferably not less than 40%. When the minimum value of the above spectral transmittances is not less than the above lower limit, the optical filter 1 when used for a sterilization device can efficiently transmit ultraviolet radiation useful for sterilization treatment.

In the optical filter 1, the maximum value of the spectral transmittances at wavelengths of 240 nm to 280 nm at an angle of incidence of 0° is preferably not more than 10%, more preferably not more than 5%, still more preferably not more than 4%, particularly preferably not more than 3%, and most preferably not more than 2%. When the maximum value of the above spectral transmittances is not more than the above upper limit, the optical filter 1 when used for a sterilization device can even further prevent transmission of ultraviolet radiation harmful to humans. The maximum value of the above spectral transmittances may be 0.01%.

In the optical filter 1, the maximum value of the spectral transmittances at wavelengths of 240 nm to 280 nm at an angle of incidence of 30° is preferably not more than 15%, more preferably not more than 10%, still more preferably not more than 5%, particularly preferably not more than 4%, and most preferably not more than 3%. When the maximum value of the above spectral transmittances is not more than the above upper limit, the optical filter 1 when used for a sterilization device can even further prevent transmission of ultraviolet radiation harmful to humans. The maximum value of the above spectral transmittances may be 0.01%.

The above spectral transmittances can be obtained, for example, by measuring the spectral transmittances of the whole optical filter 1 with a spectral transmissometer (for example, product number “UH4150” manufactured by Hitachi High-Tech Science Corporation). The above spectral transmittances mean spectral transmittances measured through the second principal surface 3b of the dielectric multi-layer film 3 in the optical filter 1. The wavelengths for the measurement can be set to a range of 190 nm to 400 nm. The above spectral transmittances are measured using the optical filter 1 in a non-curved form (flat form).

The angle of incidence means the tilt angle of incident light with respect to a normal direction (the angle represented by θ in FIG. 1) where the normal direction is a direction of layering (a direction of thickness) of the dielectric multi-layer film 3 orthogonal to a direction along the principal surface of the optical filter 1 on the dielectric multi-layer film 3 side. Therefore, the angle of incidence of 0° means a direction along the normal direction and the angle of incidence of 90° means a direction along the principal surface of the optical filter 1 on the dielectric multi-layer film 3 side.

The total thickness t_{H} and per-layer thickness of the high-refractive index films 4, the total thickness t_{L} and per-layer thickness of the low-refractive index films 5, and the total thickness of the dielectric multi-layer film 3 are not particularly limited and can be appropriately selected according to a desired light transmittance, the form of the optical filter 1 in use, and so on.

The total thickness t_{H} of the high-refractive index films 4 (the total of the respective thicknesses of the high-refractive index films 4) is preferably not less than 250 nm, more preferably not less than 300 nm, still more preferably not less than 400 nm, particularly preferably not less than 500 nm, preferably not more than 1000 nm, more preferably not more than 800 nm, still more preferably not more than 700 nm, and particularly preferably not more than 600 nm. When the total thickness t_{H} of the high-refractive index films 4 is not less than the above lower limit, the maximum value of the spectral transmittances at wavelengths of 240 nm to 320 nm can be further reduced. When the total thickness t_{H} of the high-refractive index films 4 is not more than the above upper limit, the minimum value of the spectral transmittances at wavelengths of 220 nm to 225 nm can be further increased.

The per-layer thickness of the high-refractive index films 4 is not particularly limited, but is preferably not less than 5 nm, more preferably not less than 10 nm, preferably not more than 60 nm, and more preferably not more than 50 nm.

The total thickness t_{L} of the low-refractive index films 5 (the total of the respective thicknesses of the low-refractive index films 5) is preferably not less than 500 nm, more preferably not less than 600 nm, still more preferably not less than 700 nm, particularly preferably not less than 800 nm, preferably not more than 2000 nm, more preferably not more than 1700 nm, still more preferably not more than 1500 nm, and particularly preferably not more than 1400 nm. When the total thickness t_{L} of the low-refractive index films 5 is not less than the above lower limit, the maximum value of the spectral transmittances at wavelengths of 240 nm to 320 nm can be even further reduced. When the total thickness t_{L} of the low-refractive index films 5 is not more than the above upper limit, the minimum value of the spectral transmittances at wavelengths of 220 nm to 225 nm can be even further increased.

The per-layer thickness of the low-refractive index films 5 is not particularly limited, but is preferably not less than 5 nm, more preferably not less than 10 nm, preferably not more than 90 nm, more preferably not more than 80 nm, and still more preferably not more than 60 nm.

The ratio (t_{H}/t_{L}) between the total thickness t_{H} of the high-refractive index films 4 and the total thickness t_{L} of the low-refractive index films 5 is preferably not less than 0.2, more preferably not less than 0.3, still more preferably not less than 0.4, particularly preferably not less than 0.5, most preferably not less than 0.6, preferably not more than 1, more preferably not more than 0.9, still more preferably not more than 0.8, and particularly preferably not more than 0.75. When the ratio (t_{H}/t_{L}) is not less than the above lower limit, the minimum value of the spectral transmittances at wavelengths of 220 nm to 225 nm can be even further increased. When the ratio (t_{H}/t_{L}) is not more than the above upper limit, the maximum value of the spectral transmittances at wavelengths of 240 nm to 320 nm can be even further reduced.

The total thickness of the dielectric multi-layer film 3 is not particularly limited, but is preferably not less than 800 nm, more preferably not less than 1000 nm, still more preferably not less than 1100 nm, particularly preferably not less than 1200 nm, preferably not more than 2500 nm, more preferably not more than 2200 nm, still more preferably not more than 2000 nm, and particularly preferably not more than 1900 nm. When the total thickness of the dielectric multi-layer film 3 is not less than the above lower limit, the maximum value of the spectral transmittances at wavelengths of 240 nm to 320 nm can be even further reduced. When the total thickness of the dielectric multi-layer film 3 is not more than the above upper limit, the minimum value of the spectral transmittances at wavelengths of 220 nm to 225 nm can be even further increased.

The number of layers of the films constituting the dielectric multi-layer film 3 is preferably not less than 20, more preferably not less than 25, still more preferably not less than 30, particularly preferably not less than 35, preferably not more than 100, more preferably not more than 80, still more preferably not more than 60, and particularly preferably not more than 45. When the number of layers of the films constituting the dielectric multi-layer film 3 is not less than the above lower limit, the maximum value of the spectral transmittances at wavelengths of 240 nm to 320 nm can be even further reduced. When the number of layers of the films constituting the dielectric multi-layer film 3 is not more than the above upper limit, the minimum value of the spectral transmittances at wavelengths of 220 nm to 225 nm can be even further increased.

The dielectric multi-layer film 3 preferably contains hafnium oxide crystals. More specifically, the high-refractive index films 4 constituting part of the dielectric multi-layer film 3 preferably contain hafnium oxide crystals and more preferably contain cubic hafnium oxide crystals. In this case, the minimum value of the spectral transmittances at wavelengths of 220 nm to 225 nm can be even further increased and, concurrently, the maximum value of the spectral transmittances at wavelengths of 240 nm to 280 nm can be even further reduced.

Whether or not the dielectric multi-layer film 3 contains cubic hafnium oxide crystals can be confirmed by whether or not any diffraction peak due to the (1 1 1) crystal plane derived from cubic hafnium oxide crystals is observed by X-ray diffraction measurement.

The X-ray diffraction measurement can be made by the wide-angle X-ray diffraction method. An example of an X-ray diffractometer that can be used is an X-ray diffractometer having a product name “SmartLab” and manufactured by Rigaku Corporation. Cukα rays can be used as a ray source. Also in the X-ray diffraction measurement, the whole optical filter 1 is measured through the second principal surface 3b of the dielectric multi-layer film 3.

In the present invention, in the X-ray diffraction measurement, the diffraction peak due to the (1 1 1) crystal plane derived from cubic hafnium oxide crystals is preferably larger than the diffraction peak due to the (−1 1 1) crystal plane derived from monoclinic hafnium oxide crystals. In this case, the minimum value of the spectral transmittances at wavelengths of 220 nm to 225 nm can be even further increased and, concurrently, the maximum value of the spectral transmittances at wavelengths of 240 nm to 280 nm can be even further reduced.

In the present invention, the ratio Ic/Im between the peak area intensity Ic of the diffraction peak due to the (1 1 1) crystal plane derived from cubic hafnium oxide crystals and the peak area intensity Im of the diffraction peak due to the (−1 1 1) crystal plane derived from monoclinic hafnium oxide crystals is preferably not less than 0.3, more preferably not less than 1, and still more preferably not less than 2. When the ratio Ic/Im is not less than the above lower limit, the minimum value of the spectral transmittances at wavelengths of 220 nm to 225 nm can be even further increased and, concurrently, the maximum value of the spectral transmittances at wavelengths of 240 nm to 280 nm can be even further reduced. The upper limit of the ratio Ic/Im is not particularly limited, but may be, for example, 10000.

In the present invention, an antireflection film may be provided on the second principal surface 2b of the transparent substrate 2. In this case, the transmissivity of ultraviolet radiation at wavelengths of 220 nm to 225 nm can be further increased.

The type of the antireflection film is not particularly limited and an example that can be used is a multi-layer including high-refractive index films with a relatively high refractive index and low-refractive index films with a relatively low refractive index. The multi-layer may be formed by alternately providing the high-refractive index films and the low-refractive index films in this order. An example that can be used as the above high-refractive index film is a film consisting primarily of hafnium oxide. An example of the above low-refractive index film is a film consisting primarily of silicon oxide, aluminum oxide, zirconium oxide, tin oxide, silicon nitride or others. The number of layers of the films constituting the above multi-layer may be, for example, not less than four and not more than 100.

A film other than the antireflection film may be laid on the second principal surface 2b of the transparent substrate 2 without impairing the effects of the present invention. Furthermore, a film may be provided on the second principal surface 3b of the dielectric multi-layer film 3 without impairing the effects of the present invention.

The optical filter 1 may be used in a curved form or used in a non-curved form (flat form). The optical filter 1 may be used in a curved form having an arc-shaped cross-section. The optical filter 1 may be deformable between a flat form and a curved form.

The curved form which is a form of the optical filter 1 in use is not particularly limited, but, for example, preferably has a radius of curvature of not more than 100 mm and more preferably has a radius of curvature of not more than 10 mm. The curved form which is a form of the optical filter 1 in use may be a curved form in which the dielectric multi-layer film 3 is located on a convex side or a curved form in which the dielectric multi-layer film 3 is located on a concave side.

The optical filter 1 is preferably used for a sterilization device, but can also be used in applications other than the sterilization device. For example, the optical filter 1 can be used for any heretofore known optical device in which an optical filter is used.

Hereinafter, a detailed description will be given of an example of a method for producing an optical filter 1.

### Method for Producing Optical Filter

Film-On Transparent Substrate Formation Step;

First, a transparent substrate 2 adjusted in terms of the arithmetic mean height Sa of its first principal surface 2a is prepared. Next, a dielectric multi-layer film 3 is formed on the first principal surface 2a of the transparent substrate 2. The dielectric multi-layer film 3 can be formed by alternately layering high-refractive index films 4 and low-refractive index films 5 in this order on the first principal surface 2a of the transparent substrate 2. Each of the high-refractive index films 4 and the low-refractive index films 5 can be formed by sputtering.

The temperature of the substrate in depositing the high-refractive index films 4 is preferably 300° C. or lower and more preferably 270° C. or lower. In this case, in the resultant optical filter 1, the minimum value of the spectral transmittances at wavelengths of 220 nm to 225 nm can be even further increased and, concurrently, the maximum value of the spectral transmittances at wavelengths of 240 nm to 280 nm can be even further reduced. The lower limit of the temperature of the substrate in depositing the high-refractive index films 4 may be, for example, 20° C.

The deposition of a high-refractive index film 4 can be performed, for example, using a target of a material constituting the high-refractive index film 4 and by setting the flow rate of an inert gas, such as argon gas, serving as a carrier gas at 50 sccm to 500 sccm and setting the applied power at 0.5 kW to 40 kW.

The deposition of a low-refractive index film 5 can be performed, for example, using a target of a material constituting the low-refractive index film 5 and by setting the flow rate of an inert gas, such as argon gas, serving as a carrier gas at 50 sccm to 500 sccm and setting the applied power at 0.5 kw to 40 kW.

Heat Treatment Step;

Next, the resultant film-on transparent substrate is subjected to heat treatment at a temperature of 450° C. or higher. Thus, an optical filter 1 can be obtained. Particularly, since the film-on transparent substrate is heated at a temperature of 450° C. or higher, the content of cubic hafnium oxide crystals can be made relatively large. Therefore, in the resultant optical filter 1, the minimum value of the spectral transmittances at wavelengths of 220 nm to 225 nm can be even further increased and, concurrently, the maximum value of the spectral transmittances at wavelengths of 240 nm to 280 nm can be even further reduced.

The temperature of the heat treatment of the film-on transparent substrate is preferably not lower than 450° C., more preferably not lower than 500° C., still more preferably not lower than 550° C., preferably not higher than 800° C., and more preferably not higher than 750° C. When the temperature of the heat treatment is in the above range, the minimum value of the spectral transmittances at wavelengths of 220 nm to 225 nm can be even further increased and, concurrently, the maximum value of the spectral transmittances at wavelengths of 240 nm to 280 nm can be even further reduced.

The time for the heat treatment of the film-on transparent substrate is not particularly limited, but may be, for example, not less than 10 minutes and not more than 120 minutes.

In the present invention, in X-ray diffraction measurement of the film-on transparent substrate before the heat treatment, the intensity of the diffraction peak due to the (−1 1 1) crystal plane derived from monoclinic hafnium oxide crystals is preferably low. The intensity of the diffraction peak due to the (−1 1 1) crystal plane derived from monoclinic hafnium oxide crystals is preferably at a microcrystal level and the height of the peak intensity is more preferably within the three times the height of the peak intensity of amorphous halo. In this case, the heat treatment can further increase the ratio Ic/Im between the peak area intensity Ic of the diffraction peak due to the (1 1 1) crystal plane derived from cubic hafnium oxide crystals and the peak area intensity Im of the diffraction peak due to the (−1 1 1) crystal plane derived from monoclinic hafnium oxide crystals. Therefore, in the resultant optical filter 1, the minimum value of the spectral transmittances at wavelengths of 220 nm to 225 nm can be even further increased and, concurrently, the maximum value of the spectral transmittances at wavelengths of 240 nm to 280 nm can be even further reduced.

In the present invention, the spectral transmittances at wavelengths of 220 nm to 225 nm and the spectral transmittances at wavelengths of 240 nm to 280 nm at each angle of incidence can be adjusted, for example, by not only the number of films constituting the dielectric multi-layer film 3 and the thicknesses and materials of the films, but also the heat treatment temperature of the film-on transparent substrate. Particularly depending on the heat treatment temperature of the film-on transparent substrate, the resultant optical filter 1 can even further increase the minimum value of the spectral transmittances at wavelengths of 220 nm to 225 nm and, concurrently, can even further reduce the maximum value of the spectral transmittances at wavelengths of 240 nm to 280 nm.

### Second Embodiment

FIG. 2 is a schematic cross-sectional view showing an optical filter according to a second embodiment of the present invention.

The optical filter 1A shown in FIG. 2 includes a transparent substrate 2, a dielectric multi-layer film 3, and an adhesion layer 6.

A difference between the optical filter 1 shown in FIG. 1 and the optical filter 1A shown in FIG. 2 is whether the adhesion layer 6 is present or absent therein.

Unless otherwise specified, the rest including preferred structures and configurations of the optical filter 1A are the same as those described in relation to the first embodiment (the optical filter 1).

In the optical filter 1A, the adhesion layer 6 is provided between the transparent substrate 2 and the dielectric multi-layer film 3. The adhesion layer 6 is a layer for increasing the adhesion force between the transparent substrate 2 and the dielectric multi-layer film 3. The adhesion layer 6 is provided immediately on the first principal surface 2a of the transparent substrate 2 and immediately on the first principal surface 3a of the dielectric multi-layer film 3.

The adhesion layer 6 is made of silicon oxide and contains silicon oxide. The adhesion layer 6 is a layer consisting primarily of silicon oxide. The adhesion layer 6 is a silicon oxide layer. However, the adhesion layer 6 may be a layer consisting primarily of a component other than silicon oxide. For example, the adhesion layer 6 may be a film consisting primarily of aluminum oxide, zirconium oxide or magnesium fluoride. These materials for the adhesion layer 6 may be used singly or in a combination of a plurality of them.

The material of the adhesion layer 6 is preferably the same as that of the transparent substrate 2.

It has heretofore been considered that provision of a layer made of the same material as a transparent substrate, such as a silicon oxide layer, on the transparent substrate offers no advantages because, in addition to no optical role played by the silicon oxide layer, this only increases the cost and production time. On the contrary, in the optical filter 1A, the adhesion layer 6, which is a silicon oxide layer, is purposefully placed on the transparent substrate 2, which makes it less likely that the dielectric multi-layer film 3 separates from the transparent substrate 2. The reason for this can be that since the transparent substrate 2 and the adhesion layer 6 are made of the same material, the adhesion force between the transparent substrate 2 and the adhesion layer 6 can be increased and the adhesion force between the adhesion layer 6 and the first layer of the high-refractive index films 4 can also be increased.

Therefore, in the optical filter 1A including the adhesion layer 6, the arithmetic mean height Sa of the surface of the transparent substrate 2 on the dielectric multi-layer film 3 side (the first principal surface 2a of the transparent substrate 2) and the arithmetic mean height Sa of the surface of the dielectric multi-layer film 3 on the side opposite to the transparent substrate 2 side (the second principal surface 3b of the dielectric multi-layer film 3) may not be small.

For example, in the optical filter 1A including the adhesion layer 6, the arithmetic mean height Sa of the surface of the transparent substrate 2 on the dielectric multi-layer film 3 side (the first principal surface 2a of the transparent substrate 2) may be more than 0.22 nm or may be not less than 0.30 nm.

For another example, in the optical filter 1A including the adhesion layer 6, the arithmetic mean height Sa of the surface of the dielectric multi-layer film 3 on the side opposite to the transparent substrate 2 side (the second principal surface 3b of the dielectric multi-layer film 3) may be more than 1.00 nm or may be not less than 1.50 nm.

Nevertheless, in the optical filter 1A including the adhesion layer 6, the arithmetic mean height Sa of the surface of the transparent substrate 2 on the dielectric multi-layer film 3 side (the first principal surface 2a of the transparent substrate 2) is preferably not more than 0.22 nm, more preferably not more than 0.20 nm, and still more preferably not more than 0.15 nm.

Furthermore, in the optical filter 1A including the adhesion layer 6, the arithmetic mean height Sa of the surface of the dielectric multi-layer film 3 on the side opposite to the transparent substrate 2 side (the second principal surface 3b of the dielectric multi-layer film 3) is preferably not more than 1.00 nm, more preferably not more than 0.70 nm, and still more preferably not more than 0.50 nm.

The thickness of the adhesion layer 6 may be, for example, about 10 nm to about 100 nm. In the case of use of the optical filter 1A in a curved form, the thickness of the adhesion layer 6 is preferably not less than 20 nm, more preferably not less than 30 nm, preferably not more than 70 nm, and more preferably not more than 60 nm. In the case of use of the optical filter 1A in a non-curved form (flat form), the thickness of the adhesion layer 6 is preferably not less than 20 nm, more preferably not less than 30 nm, preferably not more than 80 nm, and more preferably not more than 70 nm.

### Sterilization Device

### First Embodiment

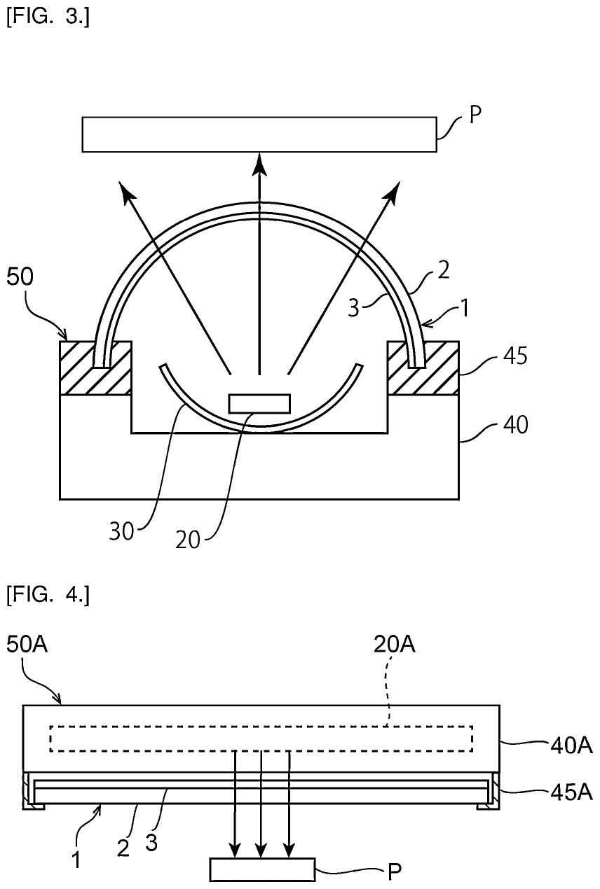

FIG. 3 is a schematic view showing a sterilization device according to a first embodiment of the present invention.

The sterilization device 50 shown in FIG. 3 is a sterilization device for subjecting a treatment object to inactivation treatment. The sterilization device 50 includes a light source 20, the optical filter 1 shown in FIG. 1, a reflector 30, a housing 40, and a retainer 45 that retains the optical filter 1 in a curved form. In the sterilization device 50, the optical filter 1 is provided in a curved form. The dielectric multi-layer film 3 is located on the concave side of the optical filter 1 and the transparent substrate 2 is located on the convex side of the optical filter 1. In the sterilization device 50, light emitted from the light source 20 is applied through the optical filter 1 to an object P to be sterilized.

The light source 20 and the reflector 30 are disposed inside the housing 40. The light source 20 is a light source the wavelength of light emitted from which is in a wavelength range of 190 nm to 230 nm. The reflector 30 can extensively diffuse the light emitted from the light source 20. The light source 20 is opposed to the concave surface of the optical filter 1.

An example of the light source 20 that can be used is an excimer lamp. A preferred excimer lamp to be used is an excimer lamp that emits ultraviolet radiation in a wavelength range of 220 nm to 225 nm. An example of this excimer lamp that can be used is a KrCl excimer lamp. The excimer lamp may be a KrBr excimer lamp.

The shape and other configurations of the retainer 45 are not particularly limited. The retainer 45 is sufficient if it can retain the optical filter 1 in a predetermined curved form. Examples of the shape of the retainer 45 include a frame shape and a box shape. The retainer may be constituted as a single separate component or may be provided, for example, as a portion of the housing.

With the use of the sterilization device 50, a treatment object (microorganisms and viruses) attached to the object P to be sterilized can be subjected to inactivation treatment. The sterilization device 50 can efficiently transmit ultraviolet radiation useful for sterilization treatment and, therefore, can efficiently sterilize the object P to be sterilized with ultraviolet radiation. For example, in ultraviolet sterilization, microorganisms, such as bacteria, can be selectively inactivated by allowing ultraviolet radiation to act on DNA in the cells of the microorganism or viruses can be selectively inactivated by allowing ultraviolet radiation to act on the viruses. Therefore, the treatment object is preferably microorganism or viruses and more preferably microorganisms. In other words, the sterilization device 50 is preferably used in order to subject microorganisms to be treated or viruses to be treated to inactivation treatment and more preferably used in order to subject microorganisms to be treated to inactivation treatment. When the optical filter 1 is provided in a curved form, the optical filter 1 can more efficiently transmit ultraviolet radiation useful for sterilization treatment and can further increase the effective irradiation area of emitted light from the light source 20.

### Second Embodiment

FIG. 4 is a schematic view showing a sterilization device according to a second embodiment of the present invention.

The sterilization device 50A shown in FIG. 4 is a sterilization device for subjecting a treatment object to inactivation treatment.

Unless otherwise specified, the rest including preferred structures and configurations of the sterilization device 50A are the same as those described in relation to the first embodiment (the sterilization device 50).

The sterilization device 50A includes a light source 20A, the optical filter 1 shown in FIG. 1, a housing 40A, and a retainer 45A that retains the optical filter 1. In the sterilization device 50A, the optical filter 1 is provided in a non-curved form (flat form). As between the transparent substrate 2 and the dielectric multi-layer film 3, the dielectric multi-layer film 3 is disposed toward the light source 20A. In the sterilization device 50A, light emitted from the light source 20A is applied through the optical filter 1 to an object P to be sterilized.

The light source 20A is a light source the wavelength of light emitted from which is in a wavelength range of 190 nm to 230 nm. The light source 20A is disposed inside the housing 40A.

An example of the light source 20A that can be used is such an excimer lamp as described previously.

The shape and other configurations of the retainer 45A are not particularly limited. The retainer 45A is sufficient if it can retain the optical filter 1. Examples of the shape of the retainer 45A include a frame shape and a box shape.

Hereinafter, a description will be given in further detail of the present invention with reference to specific examples. The present invention is not at all limited by the following examples and modifications and variations may be appropriately made therein without changing the gist of the invention.

Transparent substrates below were prepared.

Transparent substrate (A):

A fused quartz glass was cut into a size of 70 mm×52 mm×1.4 mm and each of a first principal surface and a second principal surface of the cut piece of fused quartz glass was polished 200 μm using a polishing solution containing #1200 alumina powder dispersed therein. Next, using a polishing solution containing cerium oxide powder dispersed therein, the first principal surface and the second principal surface were optically polished, thus obtaining a transparent substrate (A).

Transparent substrate (B):

A fused quartz glass was cut into a size of 70 mm×52 mm×1.4 mm and each of a first principal surface and a second principal surface of the cut piece of fused quartz glass was polished 150 μm using a polishing solution containing #1200 alumina powder dispersed therein. Next, using a polishing solution containing cerium oxide powder dispersed therein, the first principal surface and the second principal surface were optically polished, thus obtaining a transparent substrate (B).

Transparent substrate (C):

A fused quartz glass was cut into a size of 70 mm×52 mm×1.4 mm and each of a first principal surface and a second principal surface of the cut piece of fused quartz glass was polished 250 μm using a polishing solution containing #1200 alumina powder dispersed therein. Next, using a polishing solution containing cerium oxide powder dispersed therein, the first principal surface and the second principal surface were optically polished, thus obtaining a transparent substrate (C).

Transparent substrate (D):

A fused quartz glass was cut into a size of 70 mm×52 mm×1.4 mm and each of a first principal surface and a second principal surface of the cut piece of fused quartz glass was polished 180 μm using a polishing solution containing #1200 alumina powder dispersed therein. Next, using a polishing solution containing cerium oxide powder dispersed therein, the first principal surface and the second principal surface were optically polished, thus obtaining a transparent substrate (D).

### EXAMPLE 1

A dielectric multi-layer film was deposited on the first principal surface of the transparent substrate (A) by sputtering. Specifically, first, a target of hafnium was sputtered using both argon gas and oxygen gas as a carrier gas, thus depositing a hafnium oxide film (HfO₂ film) on the first principal surface of the transparent substrate (A). In doing so, each of the flow rates of argon gas and oxygen gas was set at 100 sccm and the power applied to the target (the deposition power) was set at 4 kW. Next, a target of silicon was sputtered using both argon gas and oxygen gas as a carrier gas, thus depositing a silicon oxide film (SiO₂ film) on the HfO₂ film. In doing so, each of the flow rates of argon gas and oxygen gas was set at 100 sccm and the power applied to the target (the deposition power) was set at 4 kW. By repeating the above operation, a dielectric multi-layer film was formed in which HfO₂ films and SiO₂ films were layered alternately, one layer after another, on the first principal surface of the transparent substrate (A) and which was composed of 38 layers in total, thus obtaining a film-on transparent substrate (an optical filter including a dielectric multi-layer film before being subjected to heat treatment). During the deposition, the substrate temperature was held at room temperature (20° C.). Next, the film-on transparent substrate was subjected to heat treatment at a temperature of 500° C. for 60 minutes under the air atmosphere, thus obtaining an optical filter (an optical filter including a dielectric multi-layer film after being subjected to the heat treatment).

### EXAMPLE 2

An optical filter was produced in the same manner as in Example 1, except that the transparent substrate (B) was used as a transparent substrate and a silicon oxide film (SiO₂ film) was deposited as an adhesion layer on the first principal surface of the transparent substrate (B) before a hafnium oxide film (HfO₂ film) was deposited. The silicon oxide film (SiO₂ film) was deposited by sputtering a target of silicon using both argon gas and oxygen gas as a carrier gas. In doing so, each of the flow rates of argon gas and oxygen gas was set at 100 sccm and the power applied to the target (the deposition power) was set at 4 kW.

### EXAMPLE 3

An optical filter was produced in the same manner as in Example 1 except that the transparent substrate (C) was used as a transparent substrate.

### EXAMPLE 4

An optical filter was produced in the same manner as in Example 1 except that the transparent substrate (D) was used as a transparent substrate.

### Comparative Example 1

An optical filter was produced in the same manner as in Example 1 except that the transparent substrate (B) was used as a transparent substrate.

Table 1 shows the thicknesses of the individual layers constituting the dielectric multi-layer film in the optical filters produced in Examples 1 to 4 and Comparative Example 1. Note that, in the optical filter produced in Example 2, a SiO₂ layer having a thickness of 50 nm was placed as an adhesion layer between the transparent substrate and the first layer of the HfO₂ films.

TABLE 1

| Transparent Substrate Side | | Thickness (nm) |
|---|---|---|
| 1st layer | HfO₂ | 35 |
| 2nd layer | SiO₂ | 39 |
| 3rd layer | HfO₂ | 32 |
| 4th layer | SiO₂ | 46 |
| 5th layer | HfO₂ | 34 |
| 6th layer | SiO₂ | 68 |
| 7th layer | HfO₂ | 34 |
| 8th layer | SiO₂ | 47 |
| 9th layer | HfO₂ | 30 |
| 10th layer | SiO₂ | 41 |
| 11th layer | HfO₂ | 30 |
| 12th layer | SiO₂ | 52 |
| 13th layer | HfO₂ | 42 |
| 14th layer | SiO₂ | 59 |
| 15th layer | HfO₂ | 28 |
| 16th layer | SiO₂ | 42 |
| 17th layer | HfO₂ | 29 |
| 18th layer | SiO₂ | 41 |
| 19th layer | HfO₂ | 28 |
| 20th layer | SiO₂ | 40 |
| 21st layer | HfO₂ | 29 |
| 22nd layer | SiO₂ | 40 |
| 23rd layer | HfO₂ | 29 |
| 24th layer | SiO₂ | 40 |
| 25th layer | HfO₂ | 28 |
| 26th layer | SiO₂ | 40 |
| 27th layer | HfO₂ | 28 |
| 28th layer | SiO₂ | 41 |
| 29th layer | HfO₂ | 28 |
| 30th layer | SiO₂ | 41 |
| 31st layer | HfO₂ | 28 |
| 32nd layer | SiO₂ | 41 |
| 33rd layer | HfO₂ | 28 |
| 34th layer | SiO₂ | 40 |
| 35th layer | HfO₂ | 31 |
| 36th layer | SiO₂ | 34 |
| 37th layer | HfO₂ | 31 |
| 38th layer | SiO₂ | 84 |

Air Side

### Evaluations

### Measurement of Surface Roughness

Each of the obtained optical filters was measured in terms of the surface roughnesses below with a white-light interferometer (“NewView 7300” manufactured by Zygo Corporation) in conformity with ISO 25178.

Arithmetic mean height Sa of the surface of the transparent substrate on the dielectric multi-layer film side;

Root-mean-square height Sq of the surface of the transparent substrate on the dielectric multi-layer film side;

Arithmetic mean height Sa of the surface of the dielectric multi-layer film on the side opposite to the transparent substrate side before heat treatment; and

Arithmetic mean height Sa of the surface of the dielectric multi-layer film on the side opposite to the transparent substrate side after heat treatment.

### Adhesiveness between Transparent Substrate and Dielectric Multi-Layer Film

Each of the obtained optical filters (optical filters including a dielectric multi-layer film after being subjected to heat treatment) was observed with an industrial microscope (“LV100” manufactured by Nikon Corporation) to check whether or not the dielectric multi-layer film was separated from the transparent substrate, and evaluated by the following criteria.

### Criteria for Determining Adhesiveness between Transparent Substrate and Dielectric Multi-Layer Film

◯: The dielectric multi-layer film is not separated from the transparent substrate.

X: There is any portion where the dielectric multi-layer film is separated from the transparent substrate.

The structures and results of the obtained optical filters are shown in Table 2 below.

TABLE 2

| | | | Ex. 1 | Ex. 2 | Ex. 3 | Ex. 4 | Comp. Ex. 1 |
|---|---|---|---|---|---|---|---|
| Transparent substrate | Type | — | (A) | (B) | (C) | (D) | (B) |
| Adhesion | Material | — | — | SiO₂ | — | — | — |
| layer | Thickness | nm | — | 50 | — | — | — |
| Surface | Arithmetic mean height Sa of | nm | 0.142 | 0.256 | 0.136 | 0.144 | 0.256 |
| roughness | the surface of transparent | | | | | | |
| | substrate on dielectric multi- | | | | | | |
| | layer film side | | | | | | |
| | Root-mean-square height Sq of | nm | 0.186 | 0.320 | 0.173 | 0.195 | 0.320 |
| | the surface of transparent | | | | | | |
| | substrate on dielectric multi- | | | | | | |
| | layer film side | | | | | | |
| | Arithmetic mean height Sa of | nm | 0.538 | 1.023 | 0.513 | 0.562 | 1.037 |
| | the surface of dielectric multi- | | | | | | |
| | layer film on the side opposite | | | | | | |
| | to transparent substrate side | | | | | | |
| | (Before heat treatment) | | | | | | |
| | Arithmetic mean height Sa of | nm | 0.630 | 1.294 | 0.617 | 0.643 | 1.316 |
| | the surface of dielectric multi- | | | | | | |
| | layer film on the side opposite | | | | | | |
| | to transparent substrate side | | | | | | |
| | (After heat treatment) | | | | | | |

| | | | | | | |
|---|---|---|---|---|---|---|
| Adhesiveness between transparent substrate and | — | ∘ | ∘ | ∘ | ∘ | x |
| dielectric multi-layer film | | | | | | |

### REFERENCE SIGNS LIST

1, 1A . . . optical filter

2 . . . transparent substrate

2a . . . first principal surface

2b . . . second principal surface

3a . . . first principal surface

3b . . . second principal surface

3 . . . dielectric multi-layer film

4 . . . high-refractive index film

5 . . . low-refractive index film

6 . . . adhesion layer

20, 20A . . . light source

30 . . . reflector

40, 40A . . . housing

45, 45A . . . retainer

50, 50A . . . sterilization device

P . . . object to be sterilized

1. An optical filter comprising:
a transparent substrate made of glass; and
a dielectric multi-layer film provided on the transparent substrate and containing hafnium oxide,
wherein a surface of the transparent substrate on the dielectric multi-layer film side has an arithmetic mean height Sa of 0.22 nm or less.

2. An optical filter comprising:
a transparent substrate made of glass; and
a dielectric multi-layer film provided on the transparent substrate and containing hafnium oxide,
wherein a surface of the dielectric multi-layer film on a side opposite to the transparent substrate side has an arithmetic mean height Sa of 1.00 nm or less.

3. An optical filter comprising:
a transparent substrate made of glass;
a dielectric multi-layer film containing hafnium oxide; and
an adhesion layer provided between the transparent substrate and the dielectric multi-layer film.

4. The optical filter according to claim 3, wherein the adhesion layer is a layer containing silicon oxide.

5. A sterilization device capable of subjecting microorganisms to be treated to inactivation treatment, the sterilization device comprising:
a light source a wavelength of light emitted from which is in a wavelength range of 190 nm to 230 nm; and
the optical filter according to claim 1.

6. The sterilization device according to claim 5, further comprising a retainer that retains the optical filter in a curved form.
